(19) United States
(12) Patent Application Publication
(10) Pub. No.: US 2018/0321235 A1
(43) Pub. Date: Nov. 8, 2018

(54) METHODS AND MATERIALS FOR TREATING AUTOIMMUNE CONDITIONS

(71) Applicant: Mayo Foundation for Medical Education and Research, Rochester, MN (US)

(72) Inventors: Theresa L. Wampler Muskardin, Byron, MN (US); Timothy B. Niewold, Wanamingo, MN (US)

(73) Assignee: Mayo Foundation for Medical Education and Research, Rochester, MN (US)

(21) Appl. No.: 15/773,334

(22) PCT Filed: Nov. 4, 2016
(86) PCT No.: PCT/US16/60654

§ 371 Date: May 3, 2018

Related U.S. Application Data
(60) Provisional application No. 62/250,935, filed on Nov. 4, 2015.

(51) Int. Cl. G01N 33/564; G01N 33/68

(52) U.S. Cl. CPC G01N 33/564; C07K 16/2878; G01N 2800/102; G01N 33/6866

(57) ABSTRACT

This document provides methods and materials involved in treating autoimmune conditions. For example, methods and materials for using either (a) one or more tumor necrosis factor alpha (TNF-α) inhibitors or (b) one or more Janus kinase (JAK) inhibitors to treat autoimmune conditions such as rheumatoid arthritis are provided.

### BACKGROUND

### 1. Technical Field

This document relates to methods and materials involved in treating autoimmune conditions. For example, this document provides methods and materials for using either (a) one or more tumor necrosis factor alpha (TNF-α) inhibitors or (b) one or more Janus kinase (JAK) inhibitors to treat autoimmune conditions such as rheumatoid arthritis.

### 2. Background Information

In rheumatoid arthritis, it is critical to institute effective treatment as soon as possible before damage ensues. Current treatment strategies are empirical given the lack of effective predictive markers to suggest which therapy is best for a given individual. TNF-α inhibitors are commonly used to treat rheumatoid arthritis, but responses to TNF-α inhibition are variable.

### SUMMARY

This document provides methods and materials for treating autoimmune conditions. For example, this document provides methods and materials for identifying a mammal having an autoimmune condition as having a serum IFN-β/IFN-α ratio less than 1.3 and administering one or more TNF-α inhibitors to that identified mammal to treat that autoimmune condition. This document also provides methods and materials for identifying a mammal having an autoimmune condition as having a serum IFN-β/IFN-α ratio greater than 1.3 and administering one or more JAK inhibitors to that identified mammal to treat that autoimmune condition. As described herein, mammals having an autoimmune condition and identified as having a serum IFN-β/IFN-α ratio less than 1.3 can respond favorably when treated with TNF-α inhibitors, while mammals having an autoimmune condition and identified as having a serum IFN-β/IFN-α ratio greater than 1.3 can respond poorly when treated with TNF-α inhibitors.

This document also provides methods for identifying a mammal as having an autoimmune condition that is responsive to treatment with one or more TNF-α inhibitors or one or more JAK inhibitors. For example, a serum sample obtained from a mammal having an autoimmune condition can be assessed to determine if the mammal has a serum IFN-β/IFN-α ratio less than 1.3 or greater than 1.3. Those mammals identified as having a serum IFN-β/IFN-α ratio less than 1.3 can be classified as being responsive to treatment with a TNF-α inhibitor. Those mammals identified as having a serum IFN-β/IFN-α ratio greater than 1.3 can be classified as being unresponsive to treatment with a TNF-α inhibitor and/or as being responsive to treatment with a JAK inhibitor.

In addition, this document provides methods and materials for identifying a mammal having an autoimmune condition as having classical monocyte cells with an elevated expression level of one or more of JAK1, TLR2, IFI27, IL1A, and MAVS and/or a reduced expression level of one or more of STAT2, GMCSF, TLR7, ILT7, and MYD88 and administering one or more TNF-α inhibitors to that identified mammal to treat that autoimmune condition. This document also provides methods and materials for (i) identifying a mammal having an autoimmune condition as having (a) monocyte cells with a reduced expression level or undetectable expression level of one or more of CD36 and IFIT2 and/or an elevated expression level of one or more of JAK1, IL1A, CD32a, TLR2, TGFB, TLR4, PDL1, CD11c, and TYK2; (b) classical monocytes with a reduced expression level or undetectable expression level of one or more of CD36 and IFIT2 and/or an elevated expression level of one or more of JAK1, IL1A, TLR2, IFI27, MAVS, TYK2, TLR4, CXCL9, BDCA1, CXCR3, STAT1 and STAT2; and/or (c) non-classical monocytes with a reduced expression level or undetectable expression level of one or more of STAT2, IL8, IFI27, CD64, ILT7, PKR, TLR7, and IRAK1 and/or an elevated expression level of one or more of JAK1, IL1A, CD32a, and TGFB and (ii) administering one or more TNF-α inhibitors to that identified mammal to treat that autoimmune condition. Further, this document provides methods and materials for identifying a mammal having an autoimmune condition as having classical monocyte cells with a reduced expression level of one or more of JAK1, TLR2, IFI27, IL1A, and MAVS and/or an elevated expression level of one or more of STAT2, GMCSF, TLR7, ILT7, and MYD88 and administering one or more JAK inhibitors to that identified mammal to treat that autoimmune condition.

As described herein, mammals having an autoimmune condition and identified as having classical monocyte cells with an elevated expression level of one or more of JAK1, TLR2, IFI27, IL1A, and MAVS and/or a reduced expression level of one or more of STAT2, GMCSF, TLR7, ILT7, and MYD88 can respond favorably when treated with TNF-α inhibitors, while mammals having an autoimmune condition and identified as having classical monocyte cells with a reduced expression level of one or more of JAK1, TLR2, IFI27, IL1A, and MAVS and/or an elevated expression level of one or more of STAT2, GMCSF, TLR7, ILT7, and MYD88 can respond poorly when treated with TNF-α inhibitors.

In addition, this document provides methods and materials for (i) identifying a mammal having an autoimmune condition as having (a) monocyte cells with an elevated expression level of one or more of CD36 and IFIT2 and/or a reduced expression level or undetectable expression level of one or more of JAK1, IL1A, CD32a, TLR2, TGFB, TLR4, PDL1, CD11c, and TYK2; (b) classical monocytes with an elevated expression level of one or more of CD36 and IFIT2 and/or a reduced expression level or undetectable expression level of one or more of JAK1, IL1A, TLR2, IFI27, MAVS, TYK2, TLR4, CXCL9, BDCA1, CXCR3, STAT1 and STAT2; and/or (c) non-classical monocytes with an elevated expression level of one or more of STAT2, IL8, IFI27, CD64, ILT7, PKR, TLR7, and IRAK1 and/or a reduced expression level or undetectable expression level of one or more of JAK1, IL1A, CD32a, and TGFB and (ii) administering one or more JAK inhibitors to that identified mammal to treat that autoimmune condition.

As described herein, mammals having an autoimmune condition and identified as having classical monocyte cells with an elevated expression level of one or more of JAK1, TLR2, IFI27, IL1A, and MAVS and/or a reduced expression level or undetectable expression level of one or more of STAT2, GMCSF, TLR7, ILT7, and MYD88 can respond favorably when treated with TNF-α inhibitors, while mammals having an autoimmune condition and identified as having monocyte cells with an elevated expression level of one or more of CD36 and IFIT2 and/or a reduced expression level or undetectable expression level of one or more of JAK1, IL1A, CD32a, TLR2, TGFB, TLR4, PDL1, CD11c, and TYK2; and/or classical monocytes with an elevated expression level of one or more of CD36 and IFIT2 and/or a reduced expression level or undetectable expression level of one or more of JAK1, IL1A, TLR2, IFI27, MAVS, TYK2, TLR4, CXCL9, BDCA1, CXCR3, STAT1 and STAT2; and/or non-classical monocytes with an elevated expression level of one or more of STAT2, IL8, IFI27, CD64, ILT7, PKR, TLR7, and IRAK1 and/or a reduced expression level or undetectable expression level of one or more of JAK1, IL1A, CD32a, and TGFB can respond poorly when treated with TNF-α inhibitors.

This document also provides methods for identifying a mammal as having an autoimmune condition that is responsive to treatment with one or more TNF-α inhibitors or one or more JAK inhibitors. For example, classical monocyte cells obtained from a mammal having an autoimmune condition can be assessed to determine if the mammal has classical monocyte cells with (a) an elevated expression level of one or more of JAK1, TLR2, IFI27, IL1A, and MAVS and/or a reduced expression level of one or more of STAT2, GMCSF, TLR7, ILT7, and MYD88 or (b) a reduced expression level of one or more of JAK1, TLR2, IFI27, IL1A, and MAVS and/or an elevated expression level of one or more of STAT2, GMCSF, TLR7, ILT7, and MYD88. Those mammals identified as having classical monocyte cells with an elevated expression level of one or more of JAK1, TLR2, IFI27, IL1A, and MAVS and/or a reduced expression level of one or more of STAT2, GMCSF, TLR7, ILT7, and MYD88 can be classified as being responsive to treatment with a TNF-α inhibitor. Those mammals identified as having a reduced expression level of one or more of JAK1, TLR2, IFI27, IL1A, and MAVS and/or an elevated expression level of one or more of STAT2, GMCSF, TLR7, ILT7, and MYD88 can be classified as being unresponsive to treatment with a TNF-α inhibitor and/or as being responsive to treatment with a JAK inhibitor.

This document also provides methods for identifying a mammal as having an autoimmune condition that is responsive to treatment with one or more TNF-α inhibitors or one or more JAK inhibitors. For example, monocyte cells obtained from a mammal having an autoimmune condition can be assessed to determine if the mammal has (a) monocyte cells with a reduced expression level or undetectable expression level of one or more of CD36 and IFIT2 and/or an elevated expression level of one or more of JAK1, IL1A, CD32a, TLR2, TGFB, TLR4, PDL1, CD11c, and TYK2; (b) classical monocytes with a reduced expression level or undetectable expression level of one or more of CD36 and IFIT2 and/or an elevated expression level of one or more of JAK1, IL1A, TLR2, IFI27, MAVS, TYK2, TLR4, CXCL9, BDCA1, CXCR3, STAT1 and STAT2; (c) non-classical monocytes with (i) a reduced expression level or undetectable expression level of one or more of STAT2, IL8, IFI27, CD64, ILT7, PKR, TLR7, and IRAK1 and/or an elevated expression level of one or more of JAK1, IL1A, CD32a, and TGFB, or (ii) an elevated expression level of one or more of CD36 and IFIT2 and/or a reduced expression level or undetectable expression level of one or more of JAK1, IL1A, CD32a, TLR2, TGFB, TLR4, PDL1, CD11c, and TYK2; (d) classical monocytes with an elevated expression level of one or more of CD36 and IFIT2 and/or a reduced expression level or undetectable expression level of one or more of JAK1, IL1A, TLR2, IFI27, MAVS, TYK2, TLR4, CXCL9, BDCA1, CXCR3, STAT1 and STAT2; and/or (e) non-classical monocytes with an elevated expression level of one or more of STAT2, IL8, IFI27, CD64, ILT7, PKR, TLR7, and IRAK1 and/or a reduced expression level or undetectable expression level of one or more of JAK1, IL1A, CD32a, and TGFB. Those mammals identified as having (a) monocyte cells with a reduced expression level or undetectable expression level of one or more of CD36 and IFIT2 and/or an elevated expression level of one or more of JAK1, IL1A, CD32a, TLR2, TGFB, TLR4, PDL1, CD11c, and TYK2; (b) classical monocytes with a reduced expression level or undetectable expression level of one or more of CD36 and IFIT2 and/or an elevated expression level of one or more of JAK1, IL1A, TLR2, IFI27, MAVS, TYK2, TLR4, CXCL9, BDCA1, CXCR3, STAT1 and STAT2; and/or (c) non-classical monocytes with a reduced expression level or undetectable expression level of one or more of STAT2, IL8, IFI27, CD64, ILT7, PKR, TLR7, and IRAK1 and/or an elevated expression level of one or more of JAK1, IL1A, CD32a, and TGFB can be classified as being responsive to treatment with a TNF-α inhibitor. Those mammals identified as having (a) monocyte cells with an elevated expression level of one or more of CD36 and IFIT2 and/or a reduced expression level or undetectable expression level of one or more of JAK1, IL1A, CD32a, TLR2, TGFB, TLR4, PDL1, CD11c, and TYK2; (b) classical monocytes with an elevated expression level of one or more of CD36 and IFIT2 and/or a reduced expression level or undetectable expression level of one or more of JAK1, IL1A, TLR2, IFI27, MAVS, TYK2, TLR4, CXCL9, BDCA1, CXCR3, STAT1 and STAT2; and/or (c) non-classical monocytes with an elevated expression level of one or more of STAT2, IL8, IFI27, CD64, ILT7, PKR, TLR7, and IRAK1 and/or a reduced expression level or undetectable expression level of one or more of JAK1, IL1A, CD32a, and TGFB can be classified as being unresponsive to treatment with a TNF-α inhibitor and/or as being responsive to treatment with a JAK inhibitor.

In general, one aspect of this document features a method for treating an autoimmune condition in a mammal. The method comprises, or consists essentially of, (a) identifying the mammal as having a serum IFN-β/IFN-α ratio greater than 1.3, and (b) administering a JAK inhibitor to the mammal under conditions wherein the severity of the autoimmune condition is reduced. The mammal can be a human. The autoimmune condition can be rheumatoid arthritis. The JAK inhibitor can be ruxolitinib, tofacitinib, baricitinib, or filgotinib.

In another aspect, this document features a method for treating an autoimmune condition in a mammal. The method comprises, or consists essentially of, (a) identifying the mammal as having a serum IFN-β/IFN-α ratio less than 1.3, and (b) administering a TNF-α inhibitor to the mammal under conditions wherein the severity of the autoimmune condition is reduced. The mammal can be a human. The autoimmune condition can be rheumatoid arthritis. The TNF-α inhibitor can be infliximab, adalimumab, certolizumab pegol, golimumab, or etanercept.

In another aspect, this document features a method for identifying a mammal as having an autoimmune condition susceptible to treatment with a JAK inhibitor. The method comprises, or consists essentially of, (a) determining that the mammal has a serum IFN-β/IFN-α ratio greater than 1.3, and (b) classifying the mammal as having an autoimmune condition susceptible to treatment with the JAK inhibitor. The mammal can be a human. The autoimmune condition can be rheumatoid arthritis. The JAK inhibitor can be ruxolitinib, tofacitinib, baricitinib, or filgotinib.

In another aspect, this document features a method for identifying a mammal as having an autoimmune condition susceptible to treatment with a TNF-α inhibitor. The method comprises, or consists essentially of, (a) determining that the mammal has a serum IFN-β/IFN-α ratio less than 1.3, and (b) classifying the mammal as having an autoimmune condition susceptible to treatment with the TNF-α inhibitor. The mammal can be a human. The autoimmune condition can be rheumatoid arthritis. The TNF-α inhibitor can be infliximab, adalimumab, certolizumab pegol, golimumab, or etanercept.

In another aspect, this document features a method for treating an autoimmune condition in a mammal. The method comprises, or consists essentially of, (a) identifying the mammal as having (i) monocytes that express an elevated level of one or more of CD36 and IFIT2 or a reduced level of one or more of JAK1, IL1A, CD32a, TLR2, TGFB, TLR4, PDL1, CD11c, and TYK2, (ii) classical monocytes that express an elevated level of one or more of CD36 and IFIT2 or a reduced level of one or more of JAK1, IL1A, TLR2, IFI27, MAVS, TYK2, TLR4, CXCL9, BDCA1, CXCR3, STAT1 and STAT2, or (iii) non-classical monocytes that express an elevated level of one or more of STAT2, IL8, IFI27, CD64, ILT7, PKR, TLR7, and IRAK1 or a reduced level of one or more of JAK1, IL1A, CD32a, and TGFB, and (b) administering a JAK inhibitor to the mammal under conditions wherein the severity of the autoimmune condition is reduced. The mammal can be a human. The autoimmune condition can be rheumatoid arthritis. The JAK inhibitor can be ruxolitinib, tofacitinib, baricitinib, or filgotinib.

In another aspect, this document features a method for treating an autoimmune condition in a mammal. The method comprises, or consists essentially of, (a) identifying the mammal as having CD14⁺/CD16⁻ monocytes that express a reduced level of a JAK1, TLR2, CD16, IFI27, IL1A, or MAVS nucleic acid or an elevated level of a STAT2, GMCSF, TLR7, ILT7, or MYD88 nucleic acid, and (b) administering a JAK inhibitor to the mammal under conditions wherein the severity of the autoimmune condition is reduced. The mammal can be a human. The autoimmune condition can be rheumatoid arthritis. The JAK inhibitor can be ruxolitinib, tofacitinib, baricitinib, or filgotinib.

In another aspect, this document features a method for treating an autoimmune condition in a mammal. The method comprises, or consists essentially of, (a) identifying the mammal as having (i) monocytes that express a reduced level of one or more of CD36 and IFIT2 or an elevated level of one or more of JAK1, IL1A, CD32a, TLR2, TGFB, TLR4, PDL1, CD11c, and TYK2, (ii) classical monocytes that express a reduced level of one or more of CD36 and IFIT2 or an elevated level of one or more of JAK1, IL1A, TLR2, IFI27, MAVS, TYK2, TLR4, CXCL9, BDCA1, CXCR3, STAT1 and STAT2, or (iii) non-classical monocytes that express a reduced level of one or more of STAT2, IL8, IFI27, CD64, ILT7, PKR, TLR7, and IRAK1 or an elevated level of one or more of JAK1, IL1A, CD32a, and TGFB, and (b) administering a TNF-α inhibitor to the mammal under conditions wherein the severity of the autoimmune condition is reduced. The mammal can be a human. The autoimmune condition can be rheumatoid arthritis. The TNF-α inhibitor can be infliximab, adalimumab, certolizumab pegol, golimumab, or etanercept.

In another aspect, this document features a method for treating an autoimmune condition in a mammal. The method comprises, or consists essentially of, (a) identifying the mammal as having CD14⁺/CD16⁻ monocytes that express an elevated level of a JAK1, TLR2, CD16, IFI27, IL1A, or MAVS nucleic acid or a reduced level of a STAT2, GMCSF, TLR7, ILT7, or MYD88 nucleic acid, and (b) administering a TNF-α inhibitor to the mammal under conditions wherein the severity of the autoimmune condition is reduced. The mammal can be a human. The autoimmune condition can be rheumatoid arthritis. The TNF-α inhibitor can be infliximab, adalimumab, certolizumab pegol, golimumab, or etanercept.

In another aspect, this document features a method for identifying a mammal as having an autoimmune condition susceptible to treatment with a JAK inhibitor. The method comprises, or consists essentially of, (a) determining that the mammal has (i) monocytes that express an elevated level of one or more of CD36 and IFIT2 or a reduced level of one or more of JAK1, IL1A, CD32a, TLR2, TGFB, TLR4, PDL1, CD11c, and TYK2, (ii) classical monocytes that express an elevated level of one or more of CD36 and IFIT2 or a reduced level of one or more of JAK1, IL1A, TLR2, IFI27, MAVS, TYK2, TLR4, CXCL9, BDCA1, CXCR3, STAT1 and STAT2, or (iii) non-classical monocytes that express an elevated level of one or more of STAT2, IL8, IFI27, CD64, ILT7, PKR, TLR7, and IRAK1 or a reduced level of one or more of JAK1, IL1A, CD32a, and TGFB, and (b) classifying the mammal as having an autoimmune condition susceptible to treatment with the JAK inhibitor. The mammal can be a human. The autoimmune condition can be rheumatoid arthritis. The JAK inhibitor can be ruxolitinib, tofacitinib, baricitinib, or filgotinib.

In another aspect, this document features a method for identifying a mammal as having an autoimmune condition susceptible to treatment with a JAK inhibitor. The method comprises, or consists essentially of, (a) determining that the mammal has CD14⁺/CD16⁻ monocytes that express a reduced level of a JAK1, TLR2, CD16, IFI27, IL1A, or MAVS nucleic acid or an elevated level of a STAT2, GMCSF, TLR7, ILT7, or MYD88 nucleic acid, and (b) classifying the mammal as having an autoimmune condition susceptible to treatment with the JAK inhibitor. The mammal can be a human. The autoimmune condition can be rheumatoid arthritis. The JAK inhibitor can be ruxolitinib, tofacitinib, baricitinib, or filgotinib.

In another aspect, this document features a method for identifying a mammal as having an autoimmune condition susceptible to treatment with a TNF-α inhibitor. The method comprises, or consists essentially of, (a) determining that the mammal has (i) monocytes that have a reduced expression level one or more of CD36 and IFIT2 or an elevated expression level of one or more of JAK1, IL1A, CD32a, TLR2, TGFB, TLR4, PDL1, CD11c, and TYK2, (ii) classical monocytes that have a reduced expression level of one or more of CD36 and IFIT2 or an elevated expression level of one or more of JAK1, IL1A, TLR2, IFI27, MAVS, TYK2, TLR4, CXCL9, BDCA1, CXCR3, STAT1 and STAT2; or (iii) non-classical monocytes that have a reduced expression level of one or more of STAT2, IL8, IFI27, CD64, ILT7, PKR, TLR7, and IRAK1 or an elevated expression level of one or more of JAK1, IL1A, CD32a, and TGFB, and (b) classifying the mammal as having an autoimmune condition susceptible to treatment with the TNF-α inhibitor. The mammal can be a human. The autoimmune condition can be rheumatoid arthritis. The TNF-α inhibitor can be infliximab, adalimumab, certolizumab pegol, golimumab, or etanercept.

In another aspect, this document features a method for identifying a mammal as having an autoimmune condition susceptible to treatment with a TNF-α inhibitor. The method comprises, or consists essentially of, (a) determining that the mammal has CD14⁺/CD16⁻ monocytes that express an elevated level of a JAK1, TLR2, CD16, IFI27, IL1A, or MAVS nucleic acid or a reduced level of a STAT2, GMCSF, TLR7, ILT7, or MYD88 nucleic acid, and (b) classifying the mammal as having an autoimmune condition susceptible to treatment with the TNF-α inhibitor. The mammal can be a human. The autoimmune condition can be rheumatoid arthritis. The TNF-α inhibitor can be infliximab, adalimumab, certolizumab pegol, golimumab, or etanercept.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1. Non-parametric correlation of pretreatment serum IFN-β/α activity ratio and DAS28 score at 14 weeks. Pre-TNFi=prior to TNF-α inhibition; r and p value by Spearman's rank order correlation test.

FIG. 2. Receiver-operator curve for pre-treatment IFN-β/α activity ratio predicting response by EULAR criteria at 14 weeks. Point corresponding to an IFN-β/α activity ratio of 1.3 is indicated by the arrow.

FIG. 3. Serum type I IFN activity prior to TNF-α inhibitor therapy in the combined test and validation cohort. Panel A shows pre-treatment IFN-β activity in subjects who had a good or moderate response to TNF-α inhibition at 12-14 weeks compared to those who had non-response by EULAR criteria. Panel B shows IFN-β/α activity ratio in pre-treatment sera in good or moderate responders and non-responders. Line indicates the median, error bars show the interquartile range, p-value by Mann-Whitney U test.

FIG. 4. Serum type I IFN activity measurements prior to and 4-6 weeks after starting TNF-α inhibitor therapy. Panels A, B, and C show type I IFN, IFN-β, and IFN-β/α, respectively, in patients with non-response. Panels D, E, and F show type I IFN, IFN-β, and IFN-β/α, respectively, in patients with good or moderate response. Line indicates the median, error bars show the interquartile range, p-value by Mann-Whitney U test.

FIG. 5. Serum type I IFN activity prior to TNF-α inhibitor therapy in the test cohort. Differences in total type I IFN activity and IFN-α activity between those with a good response and those with no response were not statistically significant (A, B). Pre-treatment IFN-β activity (C) and IFN-β/α activity ratio (D) were higher in those with no response at 14 weeks. Line indicates the median, error bars show the interquartile range, p-value by Mann-Whitney U test.

FIG. 6. Scatter plots showing human rheumatoid arthritis patient monocyte isolation using MACS kit (monocyte purification protocol; MiltenyiBiotec, Auburn, Calif.). The purity was greater than 95% for classical (CD14⁺/CD16⁻) and non-classical monocytes.

FIG. 7. Unsupervised hierarchical clustering of 87 target genes in single classical monocytes. This heatmap depicts gene expression data from single classical monocytes. Green represents decreased expression; red represents increased expression. Each vertical line/column represents a single classical monocyte. Each row represents a gene. Both the genes and single cells were selected for clustering. Each color in the separate bar at the bottom represents a patient. The bar at the top indicates the response/non-response group. That is, the lightest shade indicates that the cell in that column is from a patient in the non-response group (i.e., with a pre-treatment serum IFN β/α ratio >1.3. The darkest shade indicates that the cell in that column is from a patient in the response group. There is major clustering of genes that appear to align with response/non-response groups. The arrows were placed at the clusters of greatest interest, with the top one at JAK1, which appears to associate strongly with treatment response.

FIG. 8. Receivor operator curve (ROC) analysis of logistic regression models for prediction of IFN β/α ratio >1.3 (non-response to TNFi therapy). Genes that differed between IFN β/α ratio groups (i.e., TNFi treatment response groups) in categorical analysis were tested in logistic regression models. Regression models from the monocyte subsets (b and c) provided increased area under the curve in ROC analysis in comparison to the mixed monocyte model (a).

### DETAILED DESCRIPTION

This document provides methods and materials for treating an autoimmune conditions. For example, this document provides methods and materials for identifying a mammal as having a serum IFN-β/IFN-α ratio less than 1.3. Once identified as having a serum IFN-β/IFN-α ratio less than 1.3, one or more TNF-α inhibitors can be administered to that identified mammal to treat that autoimmune condition. In some cases, a mammal can be identified as having a serum IFN-β/IFN-α ratio greater than 1.3. Once identified as having a serum IFN-β/IFN-α ratio greater than 1.3, one or more JAK inhibitors can be administered to that identified mammal to treat that autoimmune condition.

This document also provides methods and materials for identifying a mammal as having classical monocyte cells with an elevated expression level of one or more of JAK1, TLR2, IFI27, IL1A, and MAVS and/or a reduced expression level of one or more of STAT2, GMCSF, TLR7, ILT7, and MYD88. Once identified as having such expression level(s), one or more TNF-α inhibitors can be administered to that identified mammal to treat that autoimmune condition. In some cases, a mammal can be identified as having classical monocyte cells with a reduced expression level of one or more of JAK1, TLR2, IFI27, IL1A, and MAVS and/or an elevated expression level of one or more of STAT2, GMCSF, TLR7, ILT7, and MYD88. Once identified as having such expression level(s), one or more JAK inhibitors can be administered to that identified mammal to treat that autoimmune condition.

This document also provides methods and materials for identifying a mammal as having (a) monocyte cells having a reduced level or undetectable level of expression of one or more of CD36 and IFIT2 and/or an elevated or detectable level of expression of one or more of JAK1, IL1A, CD32a, TLR2, TGFB, TLR4, PDL1, CD11c, and TYK2, (b) classical monocytes having a reduced level or undetectable level of expression of one or more of CD36 and IFIT2 and/or an elevated or detectable level of expression of one or more of JAK1, IL1A, TLR2, IFI27, MAVS, TYK2, TLR4, CXCL9, BDCA1, CXCR3, STAT1 and STAT2, and/or (c) non-classical monocytes having a reduced level or undetectable level of expression of one or more of STAT2, IL8, IFI27, CD64, ILT7, PKR, TLR7, and IRAK1 and/or an elevated or detectable level of expression of one or more of JAK1, IL1A, CD32a, and TGFB. Once identified as having such expression level(s), one or more TNF-α inhibitors can be administered to that identified mammal to treat that autoimmune condition. In some cases, a mammal can be identified as having (a) monocyte cells having an elevated or detectable level of expression of one or more of CD36 and IFIT2 and/or a reduced level or undetectable level of expression of one or more of JAK1, IL1A, CD32a, TLR2, TGFB, TLR4, PDL1, CD11c, and TYK2, (b) classical monocytes having an elevated or detectable level of expression of one or more of CD36 and IFIT2 and/or a reduced level or undetectable level of expression of one or more of JAK1, IL1A, TLR2, IFI27, MAVS, TYK2, TLR4, CXCL9, BDCA1, CXCR3, STAT1 and STAT2; and/or (c) non-classical monocytes having an elevated or detectable level of expression of one or more of STAT2, IL8, IFI27, CD64, ILT7, PKR, TLR7, and IRAK1 and/or a reduced level or undetectable level of expression of one or more of JAK1, IL1A, CD32a, and TGFB. Once identified as having such expression level(s), one or more JAK inhibitors can be administered to that identified mammal to treat that autoimmune condition.

Any appropriate mammal having an autoimmune condition can be treated as described herein. For example, humans and other primates such as monkeys having an autoimmune condition can be identified as having (a) a serum IFN-β/IFN-α ratio greater than 1.3 or a serum IFN-β/IFN-α ratio less than 1.3 or (b) monocyte cells with (1) an elevated expression level of one or more of JAK1, TLR2, IFI27, IL1A, and MAVS and/or a reduced expression level of one or more of STAT2, GMCSF, TLR7, ILT7, and MYD88 or (2) a reduced expression level of one or more of JAK1, TLR2, IFI27, IL1A, and MAVS and/or an elevated expression level of one or more of STAT2, GMCSF, TLR7, ILT7, and MYD88. In some cases, dogs, cats, horses, cows, pigs, sheep, mice, and rats can be identified and treated with one or more TNF-α inhibitors or one or more JAK inhibitors as described herein.

In some cases, humans and other primates such as monkeys having an autoimmune condition can be identified as having (a) a serum IFN-β/IFN-α ratio greater than 1.3 or a serum IFN-β/IFN-α ratio less than 1.3, (b) monocyte cells having a reduced level or undetectable level of one or more of CD36 and IFIT2 and/or an elevated or detectable level of one or more of JAK1, IL1A, CD32a, TLR2, TGFB, TLR4, PDL1, CD11c, and TYK2, (c) classical monocytes a reduced level or undetectable level of one or more of CD36 and IFIT2 and/or an elevated or detectable level of one or more of JAK1, IL1A, TLR2, IFI27, MAVS, TYK2, TLR4, CXCL9, BDCA1, CXCR3, STAT1 and STAT2, (d) non-classical monocytes having a reduced level or undetectable level of one or more of STAT2, IL8, IFI27, CD64, ILT7, PKR, TLR7, and IRAK1 and/or an elevated or detectable level of one or more of JAK1, IL1A, CD32a, and TGFB, (e) monocyte cells having an elevated or detectable level of one or more of CD36 and IFIT2 and/or a reduced level or undetectable level of one or more of JAK1, IL1A, CD32a, TLR2, TGFB, TLR4, PDL1, CD11c, and TYK2, (f) classical monocytes having an elevated or detectable level of one or more of CD36 and IFIT2 and/or a reduced level or undetectable level of one or more of JAK1, IL1A, TLR2, IFI27, MAVS, TYK2, TLR4, CXCL9, BDCA1, CXCR3, STAT1 and STAT2, and/or (g) non-classical monocytes having an elevated or detectable level of one or more of STAT2, IL8, IFI27, CD64, ILT7, PKR, TLR7, and IRAK1 and/or a reduced level or undetectable level of one or more of JAK1, IL1A, CD32a, and TGFB. In some cases, dogs, cats, horses, cows, pigs, sheep, mice, and rats can be identified and treated with one or more TNF-α inhibitors or one or more JAK inhibitors as described herein.

Any appropriate autoimmune condition can be assessed and treated as described herein. Examples of autoimmune conditions that can be assessed and/or treated as described herein include, without limitation, rheumatoid arthritis, Crohn's disease, juvenile idiopathic arthritis, psoriasis, and psoriatic arthritis.

Any appropriate method can be used to identify a mammal having an autoimmune condition. For example, blood tests, imaging studies, and biopsy techniques can be used to identify mammals (e.g., humans) having rheumatoid arthritis.

Once identified as having an autoimmune condition, the mammal can be assessed to determine if the mammal has a serum IFN-β/IFN-α ratio greater than 1.3 or a serum IFN-β/IFN-α ratio less than 1.3. Any appropriate method such as ELISAs, functional assays, and/or multiplex cytokine assays can be used to determine the IFN-β/IFN-α ratio of a serum sample.

Once identified as having a serum IFN-β/IFN-α ratio greater than 1.3, the mammal can be administered or instructed to self-administer one or more JAK inhibitors to treat or reduce the severity of the autoimmune condition. Examples of JAK inhibitors include, without limitation, ruxolitinib, tofacitinib, baricitinib, and filgotinib. In some cases, two or more JAK inhibitors (e.g., two, three, four, five, or more JAK inhibitors) can be administered to a mammal to treat or reduce the severity of the autoimmune condition.

Once identified as having a serum IFN-β/IFN-α ratio less than 1.3, the mammal can be administered or instructed to self-administer one or more TNF-α inhibitors to treat or reduce the severity of the autoimmune condition. Examples of TNF-α inhibitors include, without limitation, infliximab, adalimumab, certolizumab pegol, golimumab, and etanercept. In some cases, two or more TNF-α inhibitors (e.g., two, three, four, five, or more TNF-α inhibitors) can be administered to a mammal to treat or reduce the severity of the autoimmune condition.

In some cases, once identified as having an autoimmune condition, the mammal can be assessed to determine if the mammal has classical monocyte cells (e.g., CD14⁺/CD16⁻ monocytes) with an elevated expression level of one or more of JAK1, TLR2, IFI27, IL1A, and MAVS and/or a reduced expression level of one or more of STAT2, GMCSF, TLR7, ILT7, and MYD88. Any appropriate method can be used to determine the expression levels of nucleic acids within monocytes (e.g., CD14⁺/CD16⁻ monocytes). For example, mRNA-based assays such as RT-PCR can be used to assess expression of JAK1, TLR2, IFI27, IL1A, MAVS, STAT2, GMCSF, TLR7, ILT7, and/or MYD88 mRNA. In some cases, polypeptide-based assays such as antibody staining techniques or ELISAs can be performed to assess expression of JAK1, TLR2, IFI27, IL1A, MAVS, STAT2, GMCSF, TLR7, ILT7, and/or MYD88 polypeptides.

In some cases, once identified as having an autoimmune condition, the mammal can be assessed to determine if the mammal has (a) monocyte cells with a reduced level or undetectable level of one or more of CD36 and IFIT2 and/or an elevated level or detectable level of one or more of JAK1, IL1A, CD32a, TLR2, TGFB, TLR4, PDL1, CD11c, and TYK2; (b) classical monocytes with a reduced level or undetectable level of one or more of CD36 and IFIT2 and/or an elevated level or detectable level of one or more of JAK1, IL1A, TLR2, IFI27, MAVS, TYK2, TLR4, CXCL9, BDCA1, CXCR3, STAT1 and STAT2; and/or (c) non-classical monocytes with a reduced level or undetectable level of one or more of STAT2, IL8, IFI27, CD64, ILT7, PKR, TLR7, and IRAK1 and/or an elevated level or detectable level of one or more of JAK1, IL1A, CD32a, and TGFB. Any appropriate method can be used to determine the expression levels of nucleic acids within monocytes (e.g., CD14⁺/CD16⁻ monocytes). For example, mRNA-based assays such as RT-PCR can be used to assess expression of CD36, IFIT2, JAK1, IL1A, CD32a, TLR2, TGFB, TLR4, PDL1, CD11c, IFI27, MAVS, TLR4, CXCL9, BDCA1, CXCR3, STAT1, STAT2, IL8, CD64, ILT7, PKR, TLR7, IRAK1 and/or TYK2 mRNA. In some cases, polypeptide-based assays such as antibody staining techniques or ELISAs can be performed to assess expression of CD36, IFIT2, JAK1, IL1A, CD32a, TLR2, TGFB, TLR4, PDL1, CD11c, IFI27, MAVS, TLR4, CXCL9, BDCA1, CXCR3, STAT1, STAT2, IL8, CD64, ILT7, PKR, TLR7, IRAK1 and/or TYK2 polypeptides.

The expression level of JAK1, TLR2, IFI27, IL1A, MAVS, STAT2, GMCSF, TLR7, ILT7, and/or MYD88 nucleic acid can be considered elevated (a) when the measured mRNA either is expressed about 1.7 fold or more in classical monocyte cells than in healthy control cells or demonstrates a statistically significant increase over healthy control cells, or (b) when the measured polypeptide level either is expressed about 50 percent or more in classical monocyte cells than in healthy control cells or demonstrates a statistically significant increase over healthy control cells. The expression level of JAK1, TLR2, IFI27, IL1A, MAVS, STAT2, GMCSF, TLR7, ILT7, and/or MYD88 nucleic acid can be considered reduced (a) when the measured mRNA either is expressed about less (e.g., at least about 1.7 fold less) in classical monocyte cells than in healthy control cells or demonstrates a statistically significant decrease over healthy control cells, or (b) when the measured polypeptide level either is expressed about less (e.g., at least about 50 percent less) in classical monocyte cells than in healthy control cells or demonstrates a statistically significant decrease over healthy control cells.

The expression level of CD36, IFIT2, CD32a, TLR4, PDL1, CD11c, CXCL9, BDCA1, CXCR3, STAT1, CD64, PKR, IRAK1, and/or TYK2 nucleic acid can be considered elevated (a) when the measured mRNA either is expressed about 1.7 fold or more in monocyte cells than in healthy control cells or demonstrates a statistically significant increase over healthy control cells, or (b) when the measured polypeptide level either is expressed about 50 percent or more in monocyte cells than in healthy control cells or demonstrates a statistically significant increase over healthy control cells. The expression level of CD36, IFIT2, CD32a, TLR4, PDL1, CD11c, CXCL9, BDCA1, CXCR3, STAT1, CD64, PKR, IRAK1, and/or TYK2 nucleic acid can be considered reduced (a) when the measured mRNA either is expressed about less (e.g., at least about 1.7 fold less) in monocyte cells than in healthy control cells or demonstrates a statistically significant decrease over healthy control cells, or (b) when the measured polypeptide level either is expressed about less (e.g., at least about 50 percent less) in monocyte cells than in healthy control cells or demonstrates a statistically significant decrease over healthy control cells.

A human JAK1 polypeptide can have the amino acid sequence set forth in GenBank® Accession No. NP_002218.2 (GI No. 102469034) and can be encoded by the nucleic acid sequence set forth in GenBank® Accession No. NM_002227.2 (GI No. 102469033). A human TLR2 polypeptide can have the amino acid sequence set forth in GenBank® Accession No. NP_003255.2 (GI No. 19718734) and can be encoded by the nucleic acid sequence set forth in GenBank® Accession No. NM_003264.3 (GI No. 68160956). A human IFI27 polypeptide can have the amino acid sequence set forth in GenBank® Accession No. NP_001275885.1 (GI No. 572879007) and can be encoded by the nucleic acid sequence set forth in GenBank® Accession No. NM_001130080.2 (GI No. 572873826). A human IL1A polypeptide can have the amino acid sequence set forth in GenBank® Accession No. NP_000566.3 (GI No. 27894330) and can be encoded by the nucleic acid sequence set forth in GenBank® Accession No. NM_000575.4 (GI No. 940517012). A human MAVS polypeptide can have the amino acid sequence set forth in GenBank® Accession No. NP_065797.2 (GI No. 83776598) and can be encoded by the nucleic acid sequence set forth in GenBank® Accession No. NM_020746.4 (GI No. 325053645). A human STAT2 polypeptide can have the amino acid sequence set forth in GenBank® Accession No. NP_005410.1 (GI No. 4885615) and can be encoded by the nucleic acid sequence set forth in GenBank® Accession No. NM_005419.3 (GI No. 291219920). A human GMCSF polypeptide can have the amino acid sequence set forth in GenBank® Accession No. NP_000749.2 (GI No. 27437030) and can be encoded by the nucleic acid sequence set forth in GenBank® Accession No. NM_000758.3 (GI No. 371502128). A human TLR7 polypeptide can have the amino acid sequence set forth in GenBank® Accession No. NP_057646.1 (GI No. 7706093) and can be encoded by the nucleic acid sequence set forth in GenBank® Accession No. NM_016562.3 (GI No. 67944638). A human ILT7 polypeptide can have the amino acid sequence set forth in GenBank® Accession No. NP_036408.4 (GI No. 751130514) and can be encoded by the nucleic acid sequence set forth in GenBank® Accession No. NM_012276.4 (GI No. 751130513). A human MYD88 polypeptide can have the amino acid sequence set forth in GenBank® Accession No. NP_001166038.1 (GI No. 289546503) and can be encoded by the nucleic acid sequence set forth in GenBank® Accession No. NM_001172567.1 (GI No. 289546502).

Once identified as having classical monocyte cells (e.g., CD14⁺/CD16⁻ monocytes) with an elevated expression level of one or more of JAK1, TLR2, IFI27, IL1A, and MAVS and/or a reduced expression level of one or more of STAT2, GMCSF, TLR7, ILT7, and MYD88, the mammal can be administered or instructed to self-administer one or more TNF-α inhibitors to treat or reduce the severity of the autoimmune condition. In some cases, two or more TNF-α inhibitors (e.g., two, three, four, five, or more TNF-α inhibitors) can be administered to a mammal to treat or reduce the severity of the autoimmune condition.

Once identified as having (a) monocyte cells having a reduced level or undetectable level of expression of one or more of CD36 and IFIT2 and/or an elevated level or detectable level of expression of one or more of JAK1, IL1A, CD32a, TLR2, TGFB, TLR4, PDL1, CD11c, and TYK2, (b) classical monocytes having a reduced level or undetectable level of expression of one or more of CD36 and IFIT2 and/or an elevated level or detectable level of one or more of JAK1, IL1A, TLR2, IFI27, MAVS, TYK2, TLR4, CXCL9, BDCA1, CXCR3, STAT1 and STAT2, and/or (c) non-classical monocytes having a reduced level or undetectable level of expression of one or more of STAT2, IL8, IFI27, CD64, ILT7, PKR, TLR7, and IRAK1 and/or an elevated level or detectable level of one or more of JAK1, IL1A, CD32a, and TGFB, the mammal can be administered or instructed to self-administer one or more TNF-α inhibitors to treat or reduce the severity of the autoimmune condition. In some cases, two or more TNF-α inhibitors (e.g., two, three, four, five, or more TNF-α inhibitors) can be administered to a mammal to treat or reduce the severity of the autoimmune condition.

In some cases, once identified as having an autoimmune condition, the mammal can be assessed to determine if the mammal has classical monocyte cells (e.g., CD14⁺/CD16⁻ monocytes) with a reduced expression level of one or more of JAK1, TLR2, IFI27, IL1A, and MAVS and/or an elevated expression level of one or more of STAT2, GMCSF, TLR7, ILT7, and MYD88. Once identified as having classical monocyte cells (e.g., CD14+/CD16− monocytes) with a reduced expression level of one or more of JAK1, TLR2, IFI27, IL1A, and MAVS and/or an elevated expression level of one or more of STAT2, GMCSF, TLR7, ILT7, and MYD88, the mammal can be administered or instructed to self-administer one or more JAK inhibitors to treat or reduce the severity of the autoimmune condition. In some cases, two or more JAK inhibitors (e.g., two, three, four, five, or more JAK inhibitors) can be administered to a mammal to treat or reduce the severity of the autoimmune condition.

In some cases, once identified as having an autoimmune condition, the mammal can be assessed to determine if the mammal has monocyte cells having a reduced level or undetectable level of expression of one or more of CD36 and IFIT2 and/or an elevated level or detectable level of one or more of JAK1, IL1A, CD32a, TLR2, TGFB, TLR4, PDL1, CD11c, and TYK2, (b) classical monocytes having a reduced level or undetectable level of expression of one or more of CD36 and IFIT2 and/or an elevated level or detectable level of one or more of JAK1, IL1A, TLR2, IFI27, MAVS, TYK2, TLR4, CXCL9, BDCA1, CXCR3, STAT1 and STAT2, and/or (c) non-classical monocytes having a reduced level or undetectable level of expression of one or more of STAT2, IL8, IFI27, CD64, ILT7, PKR, TLR7, and IRAK1 and/or an elevated level or detectable level of one or more of JAK1, IL1A, CD32a, and TGFB. Once identified as having (a) monocyte cells having a reduced level or undetectable level of expression of one or more of CD36 and IFIT2 and/or an elevated level or detectable level of one or more of JAK1, IL1A, CD32a, TLR2, TGFB, TLR4, PDL1, CD11c, and TYK2, (b) classical monocytes having a reduced level or undetectable level of expression of one or more of CD36 and IFIT2 and/or an elevated level or detectable level of one or more of JAK1, IL1A, TLR2, IFI27, MAVS, TYK2, TLR4, CXCL9, BDCA1, CXCR3, STAT1 and STAT2, and/or (c) non-classical monocytes having a reduced level or undetectable level of expression of one or more of STAT2, IL8, IFI27, CD64, ILT7, PKR, TLR7, and IRAK1 and/or an elevated level or detectable level of one or more of JAK1, IL1A, CD32a, and TGFB, the mammal can be administered or instructed to self-administer one or more JAK inhibitors to treat or reduce the severity of the autoimmune condition. In some cases, two or more JAK inhibitors (e.g., two, three, four, five, or more JAK inhibitors) can be administered to a mammal to treat or reduce the severity of the autoimmune condition.

In some cases, one or more TNF-α inhibitors or one or more JAK inhibitors can be administered to a mammal once or multiple times over a period of time ranging from days to months. In some cases, one or more TNF-α inhibitors or one or more JAK inhibitors can be formulated into a pharmaceutically acceptable composition for administration to a mammal having an autoimmune condition. For example, a therapeutically effective amount of a JAK inhibitor can be formulated together with one or more pharmaceutically acceptable carriers (additives) and/or diluents. A pharmaceutical composition can be formulated for administration in solid or liquid form including, without limitation, sterile solutions, suspensions, sustained-release formulations, tablets, capsules, pills, powders, and granules.

Pharmaceutically acceptable carriers, fillers, and vehicles that may be used in a pharmaceutical composition described herein include, without limitation, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A pharmaceutical composition containing one or more TNF-α inhibitors or one or more JAK inhibitors can be designed for oral or parenteral (including subcutaneous, intramuscular, intravenous, and intradermal) administration. When being administered orally, a pharmaceutical composition can be in the form of a pill, tablet, or capsule. Compositions suitable for parenteral administration include aqueous and non-aqueous sterile injection solutions that can contain anti-oxidants, buffers, bacteriostats, and solutes that render the formulation isotonic with the blood of the intended recipient. The formulations can be presented in unit-dose or multi-dose containers, for example, sealed ampules and vials, and may be stored in a freeze dried (lyophilized) condition requiring only the addition of the sterile liquid carrier, for example, water for injections, immediately prior to use. Extemporaneous injection solutions and suspensions may be prepared from sterile powders, granules, and tablets.

In some cases, a pharmaceutically acceptable composition including one or more TNF-α inhibitors or one or more JAK inhibitors can be administered locally or systemically. For example, a composition provided herein can be administered locally by injection into joints. In some cases, a composition provided herein can be administered systemically, orally, or by injection to a mammal (e.g., a human).

Effective doses can vary depending on the severity of the autoimmune condition, the route of administration, the age and general health condition of the subject, excipient usage, the possibility of co-usage with other therapeutic treatments such as use of other agents, and the judgment of the treating physician.

An effective amount of a composition containing one or more TNF-α inhibitors or one or more JAK inhibitors can be any amount that reduces the severity of the autoimmune condition within the mammal without producing significant toxicity to the mammal. For example, an effective amount of a JAK inhibitor such as tofacitinib can be from about 5 mg/day to about 10 mg/day. In some cases, between about 5 mg and about 10 mg of a JAK inhibitor can be administered to an average sized human (e.g., about 75-85 kg human) between about 7 and 14 times a week. In some cases, an effective amount of a TNF-α inhibitor such as infliximab can be from about 5 mg/day to about 20 mg/day. In some cases, between about 20 mg and about 160 mg of a TNF-α inhibitor can be administered to an average sized human (e.g., about 75-85 kg human) from about one to two times a week. If a particular mammal fails to respond to a particular amount, then the amount of TNF-α inhibitor or JAK inhibitor can be increased by, for example, two fold. After receiving this higher amount, the mammal can be monitored for both responsiveness to the treatment and toxicity symptoms, and adjustments made accordingly. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and severity of the condition (e.g., autoimmune condition) may require an increase or decrease in the actual effective amount administered.

The frequency of administration of a TNF-α inhibitor or a JAK inhibitor can be any amount that reduces the severity of an autoimmune condition present within the mammal without producing significant toxicity to the mammal. For example, the frequency of administration of a TNF-α inhibitor or a JAK inhibitor can be from about two to about three times a week to about two to about three times a month. The frequency of administration of a TNF-α inhibitor or a JAK inhibitor can remain constant or can be variable during the duration of treatment. A course of treatment with a composition containing a TNF-α inhibitor or a JAK inhibitor can include rest periods. For example, a composition containing a TNF-α inhibitor or a JAK inhibitor can be administered daily over a two week period followed by a two week rest period, and such a regimen can be repeated multiple times. As with the effective amount, various factors can influence the actual frequency of administration used for a particular application. For example, the effective amount, duration of treatment, use of multiple treatment agents, route of administration, and severity of the condition (e.g., an autoimmune condition) may require an increase or decrease in administration frequency.

An effective duration for administering a composition containing one or more TNF-α inhibitors or one or more JAK inhibitors can be any duration that reduces the severity of an autoimmune condition present within the mammal without producing significant toxicity to the mammal. In some cases, the effective duration can vary from several days to several weeks, months, or years. In general, the effective duration for reducing the severity of an autoimmune condition present within the mammal can range in duration from about one month to about 10 years. Multiple factors can influence the actual effective duration used for a particular treatment. For example, an effective duration can vary with the frequency of administration, effective amount, use of multiple treatment agents, route of administration, and severity of the condition being treated.

In certain instances, a course of treatment and/or the severity of an autoimmune condition can be monitored. Any appropriate method can be used to determine whether or not the severity of an autoimmune condition present within a mammal is reduced. For example, blood tests, imaging studies, and biopsy techniques can be used to assess the severity of an autoimmune condition present within a mammal.

The invention will be further described in the following examples, which do not limit the scope of the invention described in the claims.

### EXAMPLES

### Example 1

### Serum Interferon Beta/Alpha Ratio Predicts Response to TNF-α Inhibition

### Study Cohorts

The test cohort included 32 rheumatoid arthritis patients from the Auto-immune Biomarkers Collaborative Network (ABCoN) Consortium (Liu et al., Mol. Med., 14(9-10):575-581 (2008)). The validation cohort included 92 rheumatoid arthritis patients from the Treatment Efficacy and Toxicity in Rheumatoid Arthritis Database and Repository (TETRAD registry, NCT01070121) (Ptacek et al., Arthritis Rheum., 65:S375-S375 (2013)). In the ABCON registry test cohort, all available pre-treatment patient samples that had received a TNF-inhibitor with follow up data, and had either a good response or non-response were used. Moderate response was excluded to examine the two groups that would be expected to show the largest differences. In the TETRAD validation set, all available pre-treatment samples that had received a TNF-inhibitor and had full follow up data available were tested. This included all EULAR response categories. All patients satisfied the ACR classification criteria for rheumatoid arthritis. Additional data included baseline and follow-up disease activity score (DAS), anti-CCP antibody titer, and type of TNF-α inhibitor used. All patients provided informed consent, and the study was approved by the institutional review boards at all participating institutions.

Detection of type I IFN in Rheumatoid Arthritis Patient Sera

Total serum type I IFN activity, IFN-α and IFN-β activity were measured using a functional reporter cell assay. Reporter cells (WISH cells, ATCC #CCL-25) were used to measure the ability of patient sera to cause type I IFN-induced gene expression (Hua et al., Arthritis Rheum., 54(6):1906-1916 (2006); and Niewold et al., Genes Immun., 8(6):492-502 (2007)). WISH cells were cultured with 50% patient serum for 6 hours. The cells were lysed, and cDNA was made from total cellular mRNA. Canonical type I IFN-induced gene expression (myxovirus resistance 1 (MX-1), RNA-dependent protein kinase (PKR), and IFN-induced protein with tetratricopeptide repeats 1 (IFIT-1); Kirou et al., Arthritis Rheum., 50(12):3958-3967 (2004)) was measured using qPCR. The relative expression of these three genes was standardized to healthy donor sera and summed to generate a score reflecting the ability of sera to cause IFN-induced gene expression (type I IFN activity).

### Determination of IFN-β/α Ratio

Sera with a detectable amount of type I IFN activity had additional aliquots tested following pre-incubation with anti-IFN-α (19.6 μg/mL, PBL Assay Science, Piscataway, N.J.) and anti-IFN-β (10.1 μg/mL, Chemicon, Temecula, Calif.) antibodies. The amount of inhibition of the observed type I IFN activity by anti-IFN-α antibody allowed for quantitative assessment of IFN-β activity, and that by anti-β antibody allowed for quantitative assessment of IFN-α activity. The ratio of IFN-β activity to IFN-α activity (IFN-β/α activity ratio) was then calculated for each serum sample using these data.

Those samples with no detectable total type I IFN activity were categorized as not having significant type I IFN present, and no ratio was calculated. The lower limit of detection for the WISH assay at 0.1, as values between 0 and 0.1 are within the variance of the assay. In each experiment, a positive control well was run for both IFN-α and IFN-β, to ensure that the WISH cells are appropriately responsive. Six negative control wells (WISH cells+media alone) were used to establish the expected baseline gene expression in the cells for each experiment. In addition, the anti-IFN-α and anti-IFN-β antibodies did not induce any IFN-induced gene expression in WISH cells. It was verified that the antibodies used to bind IFN-α and IFN-β could completely neutralize a significant dose of IFN-α or IFN-β respectively at the dose used, and that the antibodies did not cross-react with the other type I IFN (e.g., anti-IFN-α antibody did not bind IFN-β, etc.).

### Statistical Analysis

For comparison of quantitative data sets, Mann-Whitney U test and Spearman rank correlation tests were used. In the test set, a receiver-operator curve was used to determine the optimum cut-off for the pre-treatment IFN-β/α activity ratio (the measurement that demonstrated the strongest p-value for a difference between non-responders and good responders) as a predictor of TNF-α inhibitor response. Patients in the test set and validation set were stratified by IFN-β/α activity ratio, and the proportions of responders and non-responders in the stratified groups were compared using Chi square and odds ratios. Logistic regression models were used to test high IFN-β/α activity ratio yes/no (ratio >1.3, based upon results from the test cohort) as a predictor variable with EULAR treatment response at follow up as the outcome variable. Demographic and clinical variables were tested for interaction or confounding in these logistic regression models. Test set and validation set data were meta-analyzed using a fixed effect model with a weighted Z-scores method, following Breslow-Day testing indicating no significant heterogeneity between the two sets of data.

### Patient Characteristics

Baseline characteristics of anti-TNF-α treated rheumatoid arthritis patients classified according to EULAR response criteria are shown in Table 1. The ABCoN Consortium subjects were the test cohort, and subjects from the TETRAD registry were the validation cohort. 27 of the 32 subjects (84 percent) from the ABCoN Consortium were of European ancestry. The majority of subjects from the TETRAD registry identified as White (72 percent). 14 percent were African American, 6 percent Asian, and 1 percent Native American. 87 percent were Non-Hispanic. Overall, the test and validation sets had similar baseline characteristics. There were significant differences (p<0.05) in baseline DAS scores between the no response and moderate response groups in the validation set, and there were differences in the type of TNF-inhibitor used between groups (Table 1).

TABLE 1
Baseline characteristics of anti-tumour necrosis factor (TNF)-treated patients
with rheumatoid arthritis classified according to European League Against Rheumatism response criteria

| | Test cohort | | Validation cohort | | |
|---|---|---|---|---|---|
| | No response | Good response | No response | Moderate response | Good response |
| Patient characteristics | (n = 13) | (n = 19) | (n = 44) | (n = 30) | (n = 18) |
| Age (mean, range) | 58 (42-74) | 53 (22-83) | 53 (24-80) | 56 (25-81) | 53 (32-69) |
| Sex (F/M) | 11/2 | 13/6 | 38/6 | 26/4 | 14/4 |
| DAS28 pretreatment | 5.29±0.92 (3.87-6.79) | 4.87±0.65 (3.90-6.12) | 4.37±1.3* (1.3-7.6) | 5.89±1.4* (2.0-7.4) | 4.97±0.89 (3.4-6.8) |
| (median, SD, range) | | | | | |
| Acti-CCP positivity (n, %) | 10 (77) | 14 (74) | 28 (64) | 21 (70) | 10 (55, 6) |
| TNF inhibitor | | | | | |
| Etanercept (n, %) | 7 (54) | 10 (53) | 24 (55) | 11 (37) | 8 (44) |
| Adalimumab (n, %) | 3 (23) | 3 (16) | 16 (36) | 11 (37) | 4 (22) |
| Infliximab | 3 (23) | 6 (32) | 1 (2)*† | 6 (20)* | 3 (17)† |
| Certolizumab (n, %) | 0 | 0 | 3 (7) | 0‡ | 3 (17)‡ |
| Golimumab (n, %) | 0 | 0 | 0 | 2 (7) | 0 |

Differences were not statistically significant between groups, unless indicated
*p<0.05 between moderate and no response groups in the validation cohort.
†p<0.05 between good and no response groups in the validation cohort.
‡p<0.05 between good and no response groups in the validation cohort.
CCP, cyclic citrullinated peptide; DAS28 disease activity score 28.

Test set indicates high IFN-β/α ratio predicts anti-TNF response

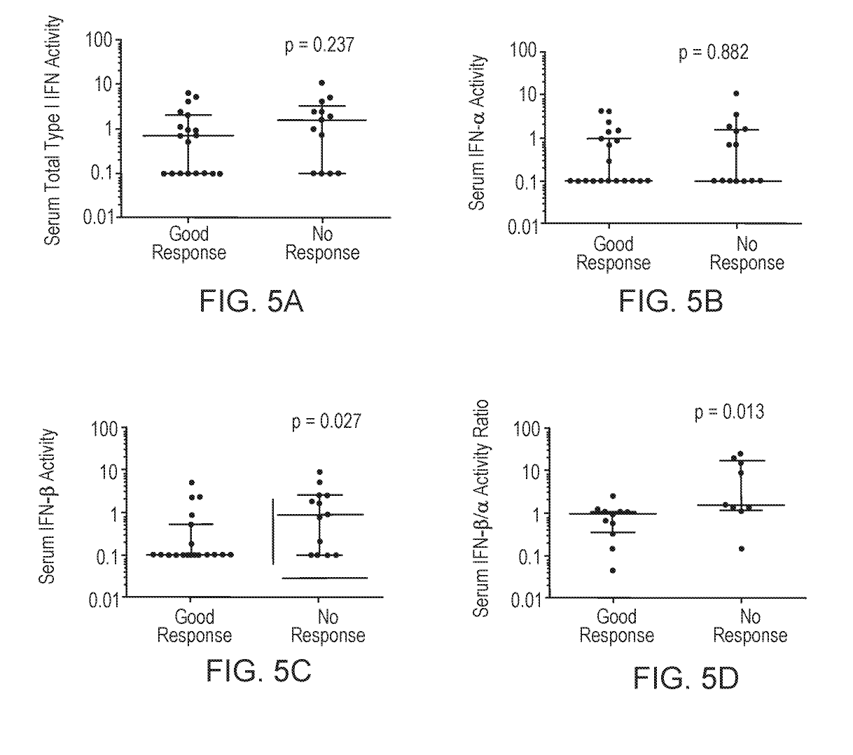

In the test cohort, type I IFN activity was tested in pre-treatment sera from 32 subjects who either had a good response or non-response to TNF-α inhibition at 14 weeks by EULAR criteria. A non-significant trend toward higher total type I IFN activity was observed in pre-treatment sera from non-responders as compared to good responders (p=0.23). When IFN-β activity was examined separately, higher pretreatment IFN-β activity was associated with lack of response to TNF-α inhibition was found (FIG. 5). There was a trend toward lower IFN-α activity in non-responders, and the IFN-β/α activity ratio between the non-responders and good responders provided the strongest p-value for a difference between groups (FIG. 5).

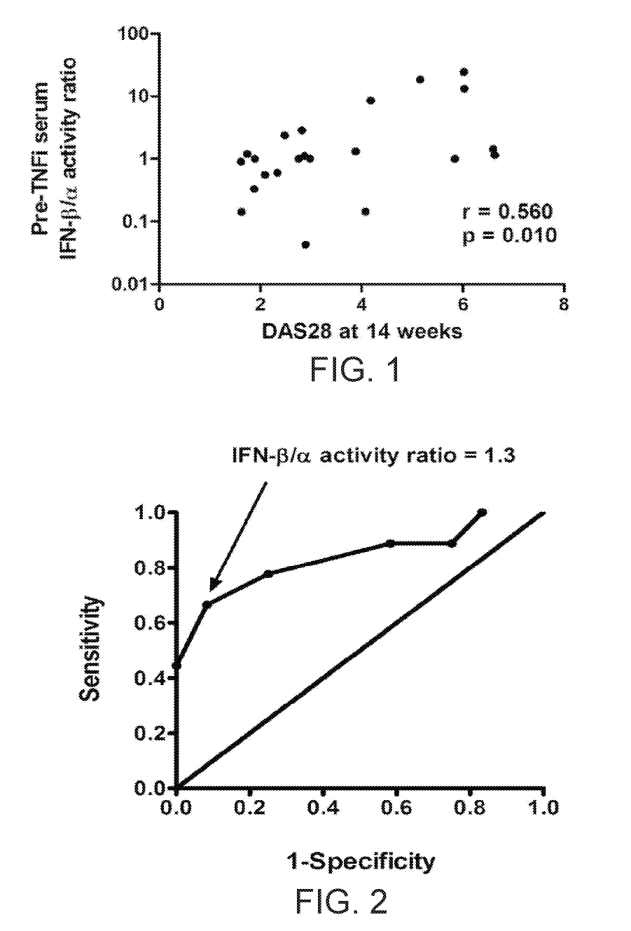

In correlation analyses, higher pre-treatment IFN-β/α activity ratio was strongly correlated with higher DAS score at 14 weeks (Spearman's rho=0.56, p=0.010, FIG. 1). Total type I IFN, IFN-β, and IFN-α activity were not significantly correlated with DAS score at 14 weeks (p>0.05 for each). In regression models, anti-CCP antibody titer and type of TNF-α inhibitor did not influence this relationship.

Based on these data, pre-treatment IFN-β/α activity ratio was selected as a predictor of response to anti-TNF-α therapy, and all TNF-α inhibitors were considered together. 12 of 32 subjects had very low total type I IFN activity. Thus, the IFN-β/α activity ratio was not determined for these subjects. A receiver-operator curve showed a strong discriminatory potential for pre-treatment IFN-β/α activity ratio, and an optimum sensitivity/specificity point at an IFN-β/α activity ratio of 1.3 (Sensitivity 67%, Specificity 92%, FIG. 2). In categorical analysis, an IFN-β/α activity ratio of >1.3 in the pretreatment sera was associated with lack of response by EULAR criteria at 14 weeks (p=0.010).

High IFN-β/α confirmed as a predictor in the validation set

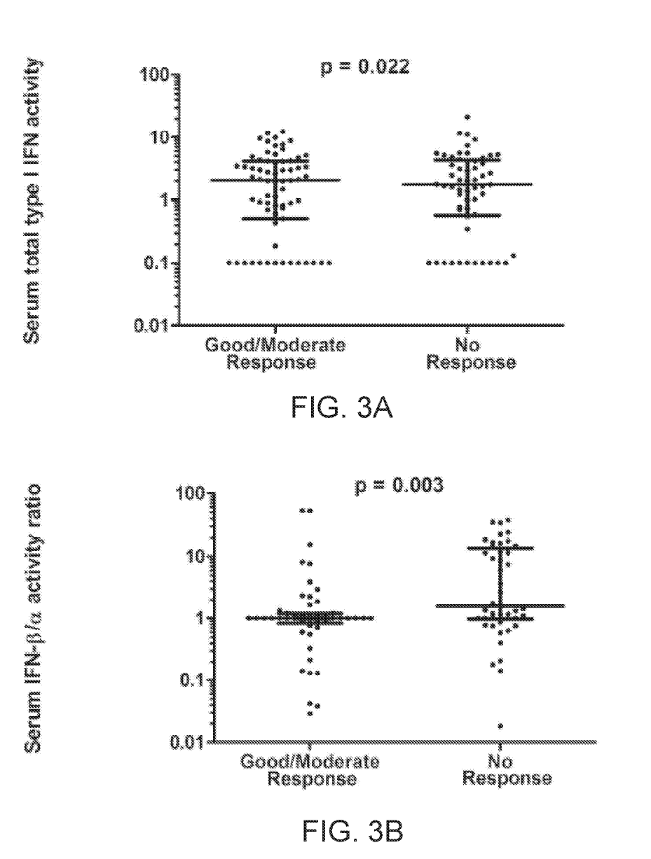

The pre-treatment IFN-β/α activity ratio cutoff point of 1.3 was tested in an independent cohort from the TETRAD consortium, consisting of pre-treatment sera from 92 subjects with good, moderate, and non-response to TNF-α inhibition at 12 weeks by EULAR criteria. IFN-β/α activity ratio could not be determined for 13 of the 92 subjects (14%), which was less than the test cohort. In the validation cohort, subjects with an IFN-β/α activity ratio >1.3 were significantly more likely to have non-response to TNF-α inhibition by EULAR criteria at 12 weeks as compared to good response (p=0.018, OR=6.67, 95% CI=1.37-32.55, Table 2). IFN-β/α activity ratio >1.3 was a significant predictor of non-response as compared to either moderate or good response (p=0.028, OR=2.80, 95% CI 1.14-6.88, Table 2), and the test had 77% specificity and 45% sensitivity for prediction of non-response as compared to moderate or good response to TNF-α-inhibition. In meta-analysis, the proportion of subjects with a pre-treatment IFN-β/α activity ratio >1.3 was significantly greater in non-responders as compared to moderate or good responders (weighted Z-score meta-analysis p-value=0.005). When baseline quantitative IFN-β/α activity ratios were compared between non-responders and good or moderate responders in the combined test and validation sets, the difference observed was greater than that observed with IFN-β alone (FIG. 3).

TABLE 2
Table 2 Categorical interferon (IFN)-β/α activity ratio by
European League Against Rheumatism (EULAR) response
criteria in the validation set

| | EULAR response | | |
|---|---|---|---|
| | category | | |
| IFN-β/α ratio | Good | Moderate | Non |
| <1.3 | 2 | 9 | 20 |
| >1.3 | 13 | 18 | 17 |
| No IFN detected | 3 | 3 | 7 |

Numbers indicate the number of subjects in each IFN-β/α activity ratio and EULAR response category.

### Follow Up Sera

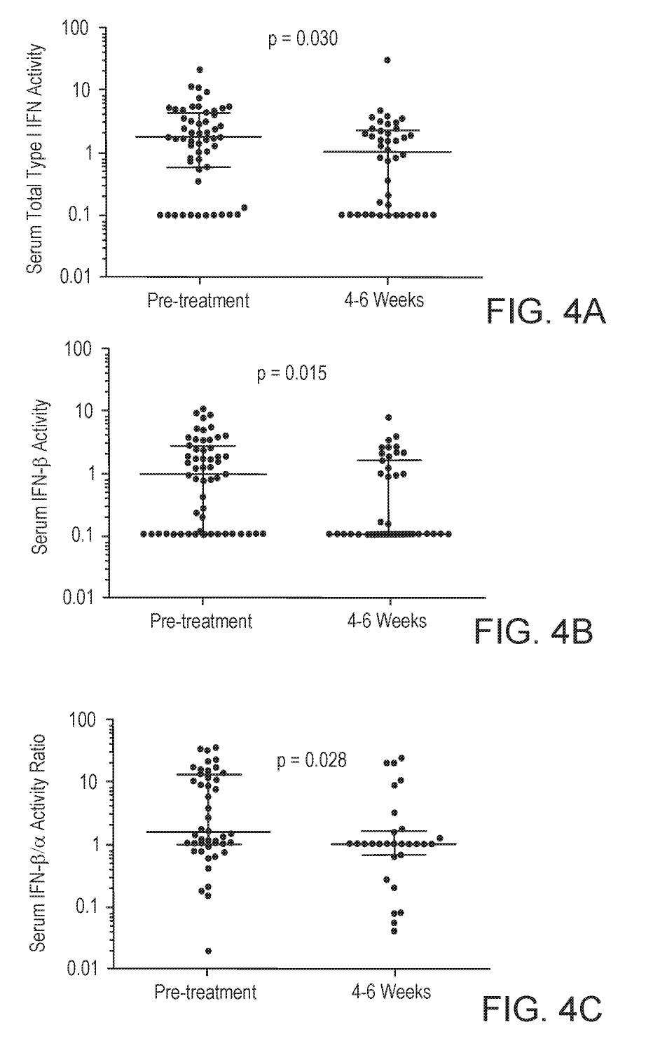

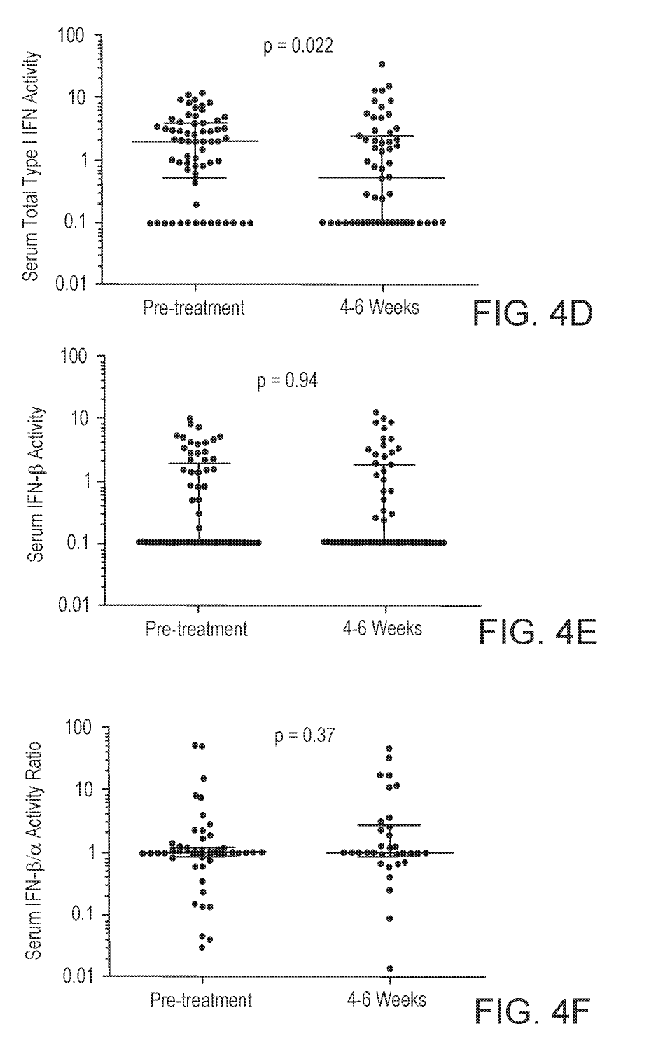

Type I IFN activity in follow up sera was also examined. In sera obtained at 4-6 weeks after starting anti-TNF-α therapy, there was a decrease in total type I IFN activity, IFN-β activity, and IFN⊕/α activity ratio (all with p value between 0.015 and 0.030, FIGS. 4A and 4C), in subjects who would later have no response at 12-14 weeks. A decrease in total type I IFN activity was observed at 4-6 weeks in the subjects who would later have a good or moderate response at 12-14 weeks (p=0.022), but no significant differences were observed in IFN-β activity or IFNβ/α activity ratio in the responders when comparing baseline sera to 4-6 week sera (FIGS. 4D-4F).

These results demonstrate that an increased pre-treatment serum IFN-0/IFN-a ratio is strongly associated with non-response to TNF-α inhibition. These results also demonstrate that a blood test designed to determine the ratio of serum IFN-β to IFN-α can be used to determine an optimal treatment option (e.g., the use of one or more TNF-α inhibitors) for treating rheumatoid arthritis or other autoimmune conditions.

### Example 2

### Determining Rheumatoid Arthritis Treatments using Gene Expression Patterns in Monocytes

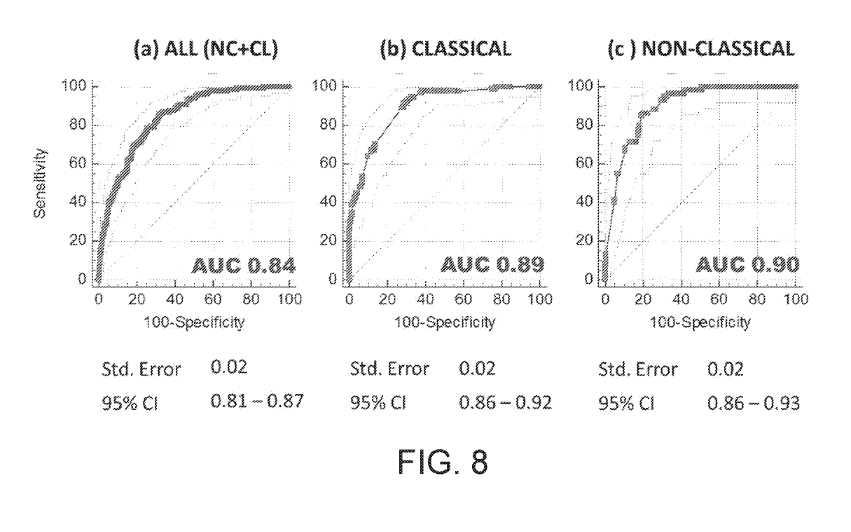

To better understand the underpinnings of the pre-treatment IFN-β/α ratio, single cell expression analysis was performed to investigate whether monocyte gene expression differs significantly between rheumatoid arthritis patients according to their pre-TNF-α inhibitor serum IFN-β/α ratio. Single classical (CL) and single non-classical (NCL) blood-derived monocytes were isolated from 15 seropositive rheumatoid arthritis subjects prior to biologic therapy. Subjects were grouped by pre-TNF-α inhibitor serum IFN-β/α ratio into two groups, IFN-β/α greater than 1.3 (n=6) and IFN-β/α less than 1.3 (n=9). Unsupervised hierarchical clustering of 87 target genes was performed to determine if there were functional gene expression differences between groups, and compared groups by Mann-Whitney and Fisher's (FIG. 7). Genes that differed in categorical analysis were tested in logistic regression models (FIG. 8).

Hierarchical clustering revealed striking differences of expression of gene sets in CL monocytes between patients with IFN-β/α less than 1.3 and IFN-β/α greater than 1.3, the groups which correspond to response/non-response to anti-TNF-α agents. This same clustering was not observed in NCL monocytes, and the differentiation between anti-TNF-α response patient groups was lost when hierarchical clustering was done on total monocytes (CL and NCL).

Two major gene sets were found to differentiate subjects with IFN-β/α greater than 1.3 (non-response to anti-TNF-α group) from those subjects with IFN-β/α less than 1.3 (response to anti-TNF-α group) in CL monocytes. The first group (Group 1) included JAK1, TLR2, CD16, IFI27, IL1A, and MAVS, with JAK1 being the most informative gene. The second group included STAT2, GMCSF, TLR7, ILT7, and MYD88, with STAT2 being the most informative gene. Subjects with IFN-β/α greater than 1.3 (non-response to anti-TNF-α group) contained CL monocytes having reduced levels of expression for the Group 1 nucleic acids and elevated levels of expression for the Group 2 nucleic acids (Table 3). Subjects with IFN-β/α less than 1.3 (response to anti-TNF-α group) contained CL monocytes having elevated levels of expression for the Group 1 nucleic acids and reduced levels of expression for the Group 2 nucleic acids (Table 3).

TABLE 3
Differential Gene Expression Rate between the treatment response groups.
Odds listed is as being expressed or not expressed in the
IFN β/α >1.3 group (TNFi non-response group).

| | | | OR | OR |
|---|---|---|---|---|
| Single Cells | Gene | P value | (Expressed) | (NOT Expressed) |
| ALL | JAK1 | <0.0001 | 0.12 | 8.19 |
| | IL1A | <0.0001 | 0.33 | 3.07 |
| | CD32a | 0.001 | 0.52 | 1.93 |
| | TLR2 | 0.007 | 0.20 | 5.12 |
| | CD36 | 0.007 | 8.95 | 0.11 |
| | IFIT2 | 0.025 | 7.08 | 0.14 |
| | TGFB | 0.031 | 0.60 | 1.68 |
| | TLR4 | 0.034 | 0.68 | 1.48 |
| | PDL1 | 0.045 | 0.69 | 1.45 |
| | CD11c | 0.046 | 0.63 | 1.59 |
| | TYK2 | 0.046 | 0.21 | 4.80 |
| CLASSICAL | JAK1 | <0.0001 | 0.07 | 15.05 |
| | IL1A | 0.001 | 0.34 | 2.91 |
| | TLR2 | 0.002 | 0.06 | 17.37 |
| | IFI27 | 0.004 | 0.43 | 2.30 |
| | MAVS | 0.013 | 0.52 | 1.94 |
| | TYK2 | 0.019 | 0.05 | 19.59 |
| | TLR4 | 0.020 | 0.56 | 1.78 |
| | CXCL9 | 0.022 | 0.47 | 2.12 |
| | BDCA1 | 0.025 | 0.50 | 1.98 |
| | CXCR3 | 0.036 | 0.48 | 2.11 |
| | IFIT2 | 0.040 | 8.88 | 0.11 |
| | CD36 | 0.041 | 9.70 | 0.10 |
| | STAT1 | 0.047 | 0.18 | 5.64 |
| | STAT2 | 0.063 | 0.58 | 1.71 |
| NON- | JAK1 | <0.0001 | 0.21 | 4.73 |
| CLASSICAL | | | | |
| | STAT2 | <0.0001 | 19.20 | 0.05 |
| | IL1A | 0.002 | 0.33 | 3.05 |
| | IL8 | 0.006 | 2.23 | 0.45 |
| | CD32a | 0.007 | 0.46 | 2.19 |
| | IFI27 | 0.012 | 2.37 | 0.42 |
| | CD64 | 0.016 | 2.00 | 0.50 |
| | ILT7 | 0.019 | 10.41 | 0.10 |
| | PKR | 0.032 | 8.92 | 0.11 |
| | TLR7 | 0.033 | 3.07 | 0.33 |
| | IRAK1 | 0.037 | 3.40 | 0.29 |
| | TGFB | 0.047 | 0.54 | 1.85 |

These results demonstrate that the expression levels of one or more nucleic acids from single monocytes (e.g., expression of CD36 and IFIT2 and non-expression of JAK1 and IL1A in the IFN β/α ratio >1.3 group) and/or single classical monocytes (e.g., expression of IFIT2 and CD36 and non-expression of TYK2, TLR2, JAK1, STAT1, and IL1A in the IFN β/α ratio >1.3 group) and/or single non-classical monocytes (e.g., expression of STAT2, ILT7, PKR, TLR7, and IRAK1 and non-expression of JAK1, IL1A, CD32a in the IFN β/α ratio >1.3 group) can be used to determine an optimal treatment option (e.g., the use of one or more TNF-α inhibitors or the use of one or more JAK inhibitors) for treating rheumatoid arthritis or other autoimmune conditions.

The results provided herein compare gene expression in monocytes from rheumatoid arthritis patients who would respond to anti-TNF agents to those rheumatoid arthritis patients who would not respond to anti-TNF therapy. The fact that JAK1 expression is the strongest differentiator between these groups supports the use of JAK inhibition in the treatment of those patients who do not respond to anti-TNF therapy.

### Example 3

### Isolation of PBMC and Monocyte Subsets

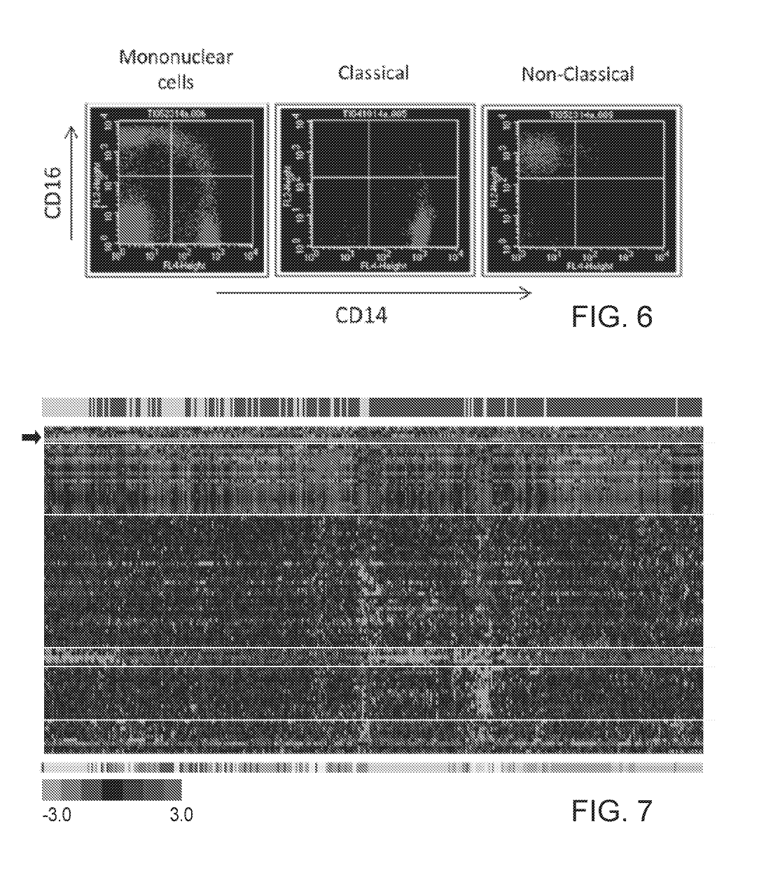

Peripheral blood mononuclear cells (PBMC) were isolated from 60 ccs of whole blood using a standard Ficoll gradient centrifugation (GE Healthcare Bio-sciences AB, Sweden). About 4×10⁶ classical (CD14⁺/CD16⁻) and non-classical (CD14⁻CD16⁺) were isolated using the MACS monocyte purification protocol (MiltenyiBiotec, Auburn, Calif.) with 95% or greater purity being achieved when assessed subsequently using flow cytometry (FIG. 6).

### Example 4

### Single Cell Gene Expression in Classical Monocytes Correlates with Treatment Response Groups to TNF-α Inhibition in Rheumatoid Arthritis

Again, in management of rheumatoid arthritis (RA), initiating effective treatment as soon as possible within the so-called therapeutic “window of opportunity” is the strategy, and disease remission is a primary goal. Pre-treatment serum type I IFN-β/α ratio >1.3 can predict non-response to anti-TNF-α therapy in RA patients. As described herein, to better understand the underpinnings of the pre-treatment IFN-β/α ratio, a single cell expression analysis was used to investigate whether monocyte gene expression differs significantly between RA patients according to their pre-TNF-α inhibitor serum IFN-β/α ratio. Single classical (CL) and single non-classical (NCL) blood-derived monocytes were isolated from 15 seropositive RA subjects prior to biologic therapy. An IFNα gene score was calculated from the expression level of 10 genes induced in healthy control blood-derived monocytes after in vitro stimulation by IFNα. An IFNβ gene score was calculated from the expression level of 10 genes found to be induced by IFNβ while excluding the possibility of influence of IFNα. Subjects were grouped by pre-TNF-α inhibitor serum IFN-β/α ratio into two groups, IFN-β/α ratio >1.3 (n=6) and IFN-β/α ratio <1.3 (n=9). Comparisons between groups were by Mann-Whitney. Hierarchical clustering of 87 target genes was done to determine if there were functional gene expression differences between groups.

Again, hierarchical clustering revealed striking differences of expression of gene sets in CL monocytes between patients with IFN-β/α ratio <1.3 and IFN-β/α ratio >1.3, the groups which correspond to response/non-response to anti-TNF-α agents. This same clustering was not observed in NCL monocytes, and the differentiation between anti-TNF-α response patient groups was lost when hierarchical clustering was done on total monocytes (CL and NCL). Two major gene sets which differentiated subjects with IFN-β/α ratio >1.3 (non-response to anti-TNF-α group) in CL monocytes included TLR and IFN pathway genes, cell surface markers and cytokines as follows: cluster 1 (GMCSF, TLR7, STAT2, ILT7, MYD88) and cluster 2 (TLR2, CD16, JAK1, IFI27, IL1A, and MAVS).

These within-cell expression patterns demonstrate biological differences in CL monocytes of RA patients with an IFN-β/α >1.3, the ratio of type I IFNs found to be predictive of non-response to anti-TNF-α therapy. Differentiation by gene expression among the response/non-response patient groups was lost when comparing gene expression in single NCL monocytes and single mixed population monocytes (CL and NCL), suggesting that further study of CL monocytes will likely illuminate molecular differences that determine treatment response to TNF-α inhibition in RA. This work can allow for a more individualized approach to therapy in RA based upon the underlying biology of disease in a given patient.

### Example 5

### Distinct Single Cell Gene Expression Signatures of Monocyte Subsets Differentiate Between TNF-α Inhibitor Treatment Response Groups in Rheumatoid Arthritis

As also described herein, to better understand the underpinnings of the pre-treatment IFN-β/α ratio, a single cell expression analysis was used to investigate whether monocyte gene expression differs significantly between RA patients according to their pre-TNF-α inhibitor serum IFN-β/α ratio. Single classical (CL) and single non-classical (NC) blood-derived monocytes were isolated from 15 seropositive RA subjects prior to biologic therapy. Subjects were grouped by pre-TNF-α inhibitor serum IFN-β/α ratio into two groups, IFN-β/α ratio >1.3 (n=6) and IFN-β/α ratio <1.3 (n=9). Unsupervised hierarchical clustering of 87 target genes was performed, and the groups were compared by Mann-Whitney and Fisher's. Genes that differed in categorical analysis were tested in logistic regression models.

Again, JAK1 and IL1A were strong differentiators between patients with IFN-β/α ratio <1.3 and IFN-β/α ratio >1.3, the groups which correspond to response/non-response to anti-TNF agents. In categorical analyses, in NC cells alone, expression (OR, p) of STAT2 (19.2, <0.0001), ILT7 (10.4, 0.02)), PKR (8.9, 0.03), TLR7 (3.1, 0.03), and IRAK1 (3.4, 0.04) was more likely in the non-response group. In CL monocytes alone, expression of IFIT2 (8.9, 0.04) and CD36 (9.7, 0.04) was more likely. In multivariate logistic regression, IL1A, CD32a, IL-8, TYK2, and IRAK1 expression in monocytes (CL+NC) aligned with treatment response groups. Each was also strongly statistically significant in regression models of monocyte subsets. In comparison to the mixed monocyte model, IL-8 and IRAK1 in NC and CXCR3 in CL cells demonstrated even stronger alignment with response groups. STAT2 was strongly predictive of response group in NC cells alone. CXCL9 was strongly predictive of response group in CL cells alone. Models from monocyte subsets provided higher area under the curve in ROC analysis in comparison to the mixed monocyte model.

These within-cell co-expression patterns demonstrate biological differences in monocyte subsets of RA patients with an IFN-β/α ratio >1.3, the ratio of type I IFNs which predicts non-response to anti-TNF therapy. Differentiation by gene expression was strongest among the response/non-response patient groups when monocyte subsets were analyzed separately, and distinct expression signatures were identified, suggesting that further study of monocyte subsets will likely illuminate molecular differences that determine treatment response to TNF-α inhibition in RA. This work can allow for a more individualized approach to therapy in RA based upon the underlying biology of disease in a given patient.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating an autoimmune condition in a mammal, wherein said method comprises:
(a) identifying said mammal as having a serum IFN-β/IFN-α ratio greater than 1.3, and
(b) administering a JAK inhibitor to said mammal under conditions wherein the severity of said autoimmune condition is reduced.

2. The method of claim 1, wherein said mammal is a human.

3. The method of claim 1, wherein said autoimmune condition is rheumatoid arthritis.

4. The method of claim 1, wherein said JAK inhibitor is ruxolitinib, tofacitinib, baricitinib, or filgotinib.

5. A method for treating an autoimmune condition in a mammal, wherein said method comprises:
(a) identifying said mammal as having a serum IFN-β/IFN-α ratio less than 1.3, and
(b) administering a TNF-α inhibitor to said mammal under conditions wherein the severity of said autoimmune condition is reduced.

6. The method of claim 5, wherein said mammal is a human.

7. The method of claim 5, wherein said autoimmune condition is rheumatoid arthritis.

8. The method of claim 5, wherein said TNF-α inhibitor is infliximab, adalimumab, certolizumab pegol, golimumab, or etanercept.

9. A method for identifying a mammal as having an autoimmune condition susceptible to treatment with a JAK inhibitor, wherein said method comprises:
(a) determining that said mammal has a serum IFN-β/IFN-α ratio greater than 1.3, and
(b) classifying said mammal as having an autoimmune condition susceptible to treatment with said JAK inhibitor.

10. The method of claim 9, wherein said mammal is a human.

11. The method of claim 9, wherein said autoimmune condition is rheumatoid arthritis.

12. The method of claim 9, wherein said JAK inhibitor is ruxolitinib, tofacitinib, baricitinib, or filgotinib.

13. A method for identifying a mammal as having an autoimmune condition susceptible to treatment with a TNF-α inhibitor, wherein said method comprises:
(a) determining that said mammal has a serum IFN-β/IFN-α ratio less than 1.3, and
(b) classifying said mammal as having an autoimmune condition susceptible to treatment with said TNF-α inhibitor.

14. The method of claim 13, wherein said mammal is a human.

15. The method of claim 13, wherein said autoimmune condition is rheumatoid arthritis.

16. The method of claim 13, wherein said TNF-α inhibitor is infliximab, adalimumab, certolizumab pegol, golimumab, or etanercept.

17-48. (canceled)
